(19) United States
(12) Patent Application Publication
(10) Pub. No.: US 2007/0014795 A1
(43) Pub. Date: Jan. 18, 2007

(54) Compositions and methods for enhanced dendritic cell maturation and function

(76) Inventors: Madhav V. Dhodapkar, New York, NY (US); Jeffrey V. Ravetch, New York, NY (US); Ralph M. Steinman, Westport, CT (US); Kavita M. Dhodapkar, New York, NY (US)

Correspondence Address: Pearl Cohen Zedek Latzer, LLP;Suite 1001, 10 Rockefeller Plaza, New York, NY, 10020, US

(21) Appl. No.: 11/317,892

(22) Filed: Dec. 27, 2005

Related U.S. Application Data
(60) Provisional application No. 60/640,091, filed on Dec. 30, 2004.

(51) Int. Cl. A61K 39/395; A61K 35/14

(52) U.S. Cl. 424/144.1; 424/93.7

(57) ABSTRACT

This invention relates to compositions comprising an agent, which inhibits signaling via the FcγRIIB receptor and an agent which stimulates or enhances signaling via an FcγRI receptor, an FcγRIIa receptor, an FcγRIII receptor, or a combination thereof. The invention also provides for the use of such compositions in stimulating or enhancing an immune response, and in treating, suppressing, or preventing cancer in a subject.

### CROSS-REFERENCE TO RELATED APPLICATIONS

This Application claims the benefit of U.S. Provisional Application Ser. No. 60/640,091, filed Dec. 30, 2004, which is hereby incorporated in its entirety.

### FIELD OF THE INVENTION

This invention relates to compositions comprising an agent, which inhibits signaling through the FcγRIIB receptor and an agent, which stimulates or enhances signaling through other FcγR receptors and methods of utilizing the same, in maturing and/or activating dendritic cells. This invention provides methods of stimulating or enhancing immune responses, and provides applications in treating or preventing infection or neoplasia.

### BACKGROUND OF THE INVENTION

Dendritic cells (DCs) are highly differentiated antigen presenting cells that play a key role in the initiation and regulation of T cell immunity to pathogens and tumors, while at the same time preventing immune responses against self-tissues or environmental antigens. A critical property of DCs is that their ability to activate or inhibit immunity is linked to environmental stimuli, which determine their final differentiation or maturation status. Several environmental (e.g. pathogen recognition via toll like receptors, CD40L on different cell types), and endogenous stimuli (e.g. heat shock proteins, inflammatory cytokines, innate lymphocytes) can lead to DC maturation and T cell immunity. However under steady state, DCs must avoid inappropriate activation in order to prevent responses to self-antigens (“horror autotoxicus”) and harmless environmental antigens. Specific pathways that prevent spontaneous DC activation are not well understood.

Circulating immune complexes and cell-bound immunoglobulins present in normal human sera represents a potential stimulus for inadvertent DC activation in the steady state. The physiologic consequences of cell bound IgG and immune complexes are modulated by a balance between activating and inhibitory Fcγ receptors (FcγRs) and include immune regulatory and inflammatory responses. Engagement of activating FcγRs that contain an immune tyrosine based activation motif (ITAM) on effector cells including monocytes, neutrophils, NK cells and mast cells, mediates phagocytosis, antibody dependent cell mediated cytotoxicity (ADCC), and release of cytokines and other inflammatory mediators. In contrast, inhibitory FcγRs contain an immune tyrosine inhibitory motif (ITIM). Signaling via these receptors leads to recruitment and phosphorylation of an SH2 domain containing, inositol polyphosphate 5 phosphatase (SHIP) that regulates signaling by activating receptors.

Recent studies have implied an important maturation role for FcγR expression on antigen presenting cells (APCs) including DCs. In addition, targeting of antigens, including immune complexes and antibody coated tumor cells to FcγRs on human DCs leads to cross-priming of both CD4+ and CD8+ T cell responses in culture.

The FcγR system represents a balance of activating and inhibitory receptors that determines the outcome of immune complex mediated inflammation and immunity. Targeting immune complexes to DCs in mice genetically lacking inhibitory FcγRIIB can lead to enhanced generation of antigen specific CD8+ T cell immunity in vitro and in vivo however, genetic deletion of FcγRIIB leads to spontaneous autoimmunity in genetically prone mice. Further confounding the issue is the fact that the FcγR system, i.e., both the number and type of activating and inhibitory receptors, differs significantly between mice and humans, and methods other than genetic deletion are required to manipulate the balance between activating and inhibitory FcγR.

### SUMMARY OF THE INVENTION

This invention provides, in one embodiment, composition for stimulating or enhancing an immune response, comprising an agent which inhibits signaling via the FcγRIIB receptor and an agent, which stimulates or enhances signaling via an FcγRI receptor, an FcγRIIa receptor, an FcγRIII receptor, or a combination thereof.

In one embodiment, the agent, which inhibits signaling via the FcγRIIB receptor, is a neutralizing antibody. In another embodiment, the agent, which stimulates or enhances signaling via an FcγRI receptor, an FcγRIIa receptor, an FcγRIII receptor, or combination thereof, is an immune complex. In one embodiment, the immune complex comprises a polypeptide or peptide, which is bound to an antibody or antibody fragment. In another embodiment, the agent, which stimulates or enhances signaling via an FcγRI receptor, an FcγRIIa receptor, an FcγRIII receptor, or combination thereof, is an antibody or antibody fragment, comprising an Fc portion which binds to the FcγRI receptor, FcγRIIa receptor, FcγRIII receptor, or combination thereof. In another embodiment, the antibody or antibody fragment is further bound to a cell.

In another embodiment, this invention provides a method for producing an isolated, differentiated dendritic cell population, comprising contacting an immature dendritic cell with an agent which inhibits signaling via the FcγRIIB receptor and an agent which stimulates or enhances signaling via an FcγRI receptor, an FcγRIIa receptor, an FcγRIII receptor, or a combination thereof and isolating the dendritic cell, whereby the isolated dendritic cell exhibits a more differentiated phenotype than the immature dendritic cell.

In another embodiment, this invention provides a method for stimulating or enhancing an immune response in a subject, comprising the steps of contacting an antigen presenting cell with an agent which inhibits signaling via the FcγRIIB receptor and an agent which stimulates or enhances signaling via an FcγRI receptor, an FcγRIIa receptor, an FcγRIII receptor, or a combination thereof whereby the antigen presenting cell contacts a T lymphocyte and the T lymphocyte stimulates or enhances an immune response in the subject, thereby being a method for stimulating or enhancing an immune response in a subject.

According to this aspect of the invention, and in one embodiment, the antigen presenting cell is a dendritic cell. In one embodiment, the antigen presenting cells are contacted in vivo, or, in another embodiment, ex vivo, with the agents, lymphocytes, or combination thereof.

In one embodiment, this invention provides a method for treating, suppressing, or preventing cancer in a subject, the method comprising the steps of contacting an immature dendritic cell with an agent which inhibits signaling via the FcγRIIB receptor and an agent, which stimulates or enhances signaling via an FcγRI receptor, an FcγRIIa receptor, an FcγRIII receptor, or a combination thereof, whereby the dendritic cell contacts a T lymphocyte and the T lymphocyte stimulates or enhances an immune response against a cancer in the subject, thereby being a method for treating, suppressing, or preventing cancer in a subject.

### BRIEF DESCRIPTION OF THE DRAWINGS

FIG. 1 demonstrates expression of FcγRIIA and FcγRIIB on human monocyte derived DCs. A. Purified CD14+ monocytes were induced to differentiate into DCs in the presence of GM-CSF and IL-4. On day 6 of culture, inflammatory cytokines were added to yield mature DCs. Expression of FcγRIIA and FcγRIIB on immature and mature DCs was determined by flow cytometry using specific antibodies (IV.3 and 2B6, respectively). Data are representative of 2 similar experiments. B. Ratio of Mean Fluorescence Intensity (MFI) of staining for FcγRIIB and RIIA. Data are representative of 2 similar experiments. C. Expression of FcgRIIa and FcgRIIb on myeloid and plasmacytoid subsets of human blood-derived DCs. Myeloid (Lin−, DR+, CD11c+) DCs and plasmacytoid (Lin− DR+ CD123+/BDCA2+) DCs were isolated from PBMCs as described under methods. Expression of FcγRIIa and FcγRIIb on immature and mature DCs was determined by flow cytometry using specific antibodies (IV.3 and 2B6, respectively). Data are representative of 3 experiments.

FIG. 2 demonstrates that blockade of FcγRIIB in the presence of human serum leads to maturation of human monocyte derived DCs. A. Monocyte derived DCs cultured in RPMI with 1% plasma, or serum free media (AIM-V) were incubated overnight with anti-FcγRIIB antibody (2B6; 1 μg/ml), or isotype control antibody. Expression of HLA-DR, CD80, CD86 and CD83 on CD11c+ DCs was monitored by flow cytometry. Data are representative of 3 similar experiments. B. Monocyte derived DCs were cultured either in serum free medium (AIM-V) or AIM-V supplemented with 1% plasma. DCs were cultured with chimeric (ch-2B6) anti-FcγRIIB antibody, or isotype control. DC maturation was monitored by flow cytometry. Data are mean/SD of 2 similar experiments. C. Representative FACS plot showing expression of maturation marker CD83/CD80 in DCs cultured under conditions described in FIG. 2b. % CD83+ cells are noted. D. Monocyte derived DCs were cultured either in RPMI with 1% plasma, or RPMI with 1% Ig depleted plasma. DCs were cultured with chimeric (ch-2B6) anti-FcγRIIB antibody, or isotype control. DC maturation was monitored by flow cytometry. Data shown are mean/SD of 2 similar experiments. Inset of western blot shows depletion of Ig from plasma Lane 1 is RPMI with 1% plasma and lane 2 is RPMI with 1% Ig depleted plasma E. Representative FACS plot showing expression of maturation marker CD83/CD80 in DCs cultured under conditions described in FIG. 2d, with isotype control, chimeric (ch-2B6) or aglycosylated anti-FcγRIIB (agly-2B6) antibody. % CD83+ cells are noted.

FIG. 3 demonstrates FcγRIIB blockade leads to IL-12p70 production. Supernatants of immature monocyte derived DCs, treated overnight with anti-FcγRIIB (2B6; 1 microgramg/ml), isotype-matched control antibody, or inflammatory cytokines, were analyzed for IL-12p70 production by ELISA.

FIG. 4 demonstrates the effect of FcγRIIb blockade on the uptake of tumor cells by DCs. Myeloma cells were labeled with dye (PKH-26), opsonized with anti-syndecan-1 antibody and cocultured with dye (PKH-67) labeled DCs at 4° C. or 37° C. After 4-8 hours of co-culture, the percentage of double positive DCs was evaluated by flow cytometry.

FIG. 5 demonstrates the effect of FcγRIIB blockade on the expansion of myeloma reactive T cells by tumor loaded DCs. A. Monocyte derived DCs alone, or loaded with opsonized U266 tumor cells, were either left untreated (no maturation cytokine) or matured ex vivo using a cytokine cocktail as a maturation stimulus (with maturation cytokines). DCs were also pretreated with either isotype control or with anti-FcγRIIB antibody (2B6). The tumor loaded and unpulsed DCs were each used to stimulate autologous T cells. Interferon-γ producers against U266 (A2+) or cag (A2−) cells as control, were analyzed by Elispot assay. Data shown are mean/SD of 3 separate experiments. B. Immature monocyte derived DCs from HLA-A2+ donors were loaded with opsonized cag (A2−) myeloma cells in the presence of isotype control or anti-FcγRIIB antibody (2B6), and used to stimulate autologous T cells. After 14 days of culture, T cells were stimulated overnight in Elispot plates with autologous DCs pulsed with 10 μM A2 restricted peptides derived from MAGE-A3, NY-ESO-1, or 2.5 μM of an overlapping 15-mer peptide library derived from survivin. Interferon-y producers were quantified by an Elispot assay. Data shown are mean/SD of independent experiments on two blood donors. *=p<0.05.

### DETAILED DESCRIPTION OF THE PRESENT INVENTION

This invention provides, in one embodiment, a composition for stimulating or enhancing an immune response, comprising an agent which inhibits signaling via the FcγRIIB receptor and an agent, which stimulates or enhances signaling via an FcγRI receptor, an FcγRIIa receptor, an FcγRIII receptor, or a combination thereof

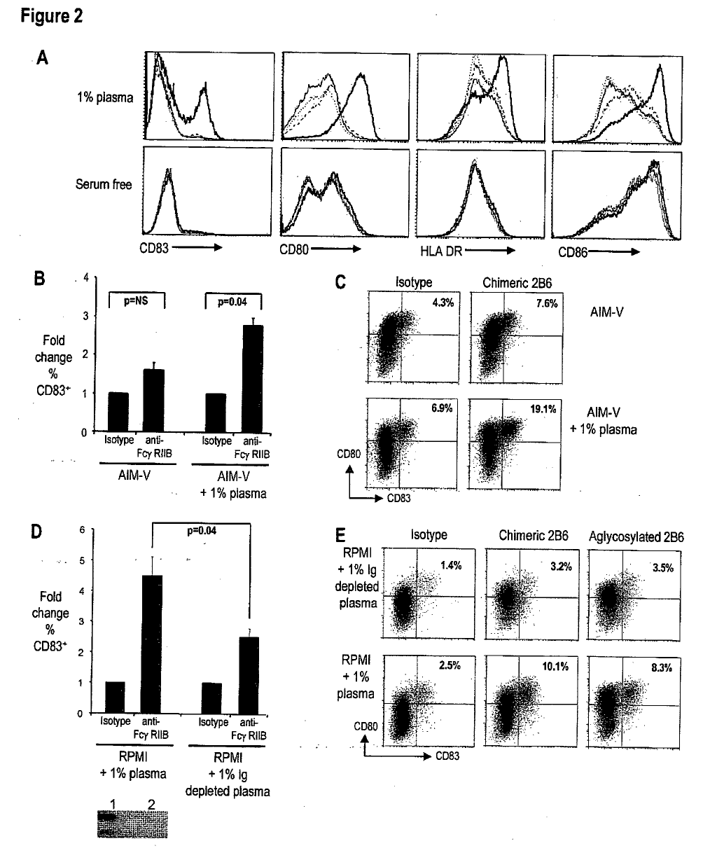

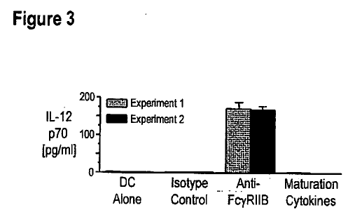

Selective blockade of the inhibitory Fcγ receptor (FcγRIIB), using monoclonal antibodies, led to maturation of human monocyte-derived dendritic cells (DCs), and was dependent on the presence of IgG in the cell cultures. DC maturation was evidenced by the upregulated expression of costimulatory molecules (FIG. 2) and production of IL-12 p70 (FIG. 3).

In one embodiment, the agent, which inhibits signaling via the FcγRIIB receptor is an antibody specifically directed against the FcγRIIB receptor. In one embodiment, the antibody is monoclonal, or in another embodiment, the antibody is polyclonal, or in another embodiment, an agent may be an antibody fragment, which when bound to a cell expressing the FcγRIIB receptor, prevents signaling through the receptor.

In one embodiment, the invention encompasses antibodies, which are, in one embodiment, monoclonal antibodies or fragments thereof that specifically bind FcγRIIB. In one embodiment, the antibodies have a high affinity for human FcγRIIB, and in another embodiment, bind native human FcγRIIB with a greater affinity than the antibodies or fagments thereof bind FcγRIIA.

In one embodiment, the antibodies comprising the compositions of this invention, or used in the methods of this invention, may include, but are not limited to, monoclonal antibodies, synthetic antibodies, recombinantly produced antibodies, multispecific antibodies, human antibodies, humanized antibodies, chimeric antibodies, camelized antibodies, single-chain Fvs (scFv), single chain antibodies, Fab fragments, F(ab′) fragments, disulfide-linked Fvs (sdFv), intrabodies, and epitope-binding fragments of any of the above. In one embodiment, antibodies used in the compositions and for the methods of the present invention include immunoglobulin molecules and immunologically active portions of immunoglobulin molecules, i.e., molecules that contain an antigen binding site that immunospecifically binds to FcγRIIB with greater affinity than the immunoglobulin molecule binds FcγRIIA. It is to be understood that antibodies, which are used for inhibiting or stimulating specific Fcγ receptors, will have an increased affinity for the specified receptor, as compared to other Fcγ receptors.

The antibodies used in the compositions and methods of the invention may be from any animal origin including birds and mammals (e.g., human, non-human primate, murine, donkey, sheep, rabbit, goat, guinea pig, camel, horse, or chicken). In one embodiment, the antibodies are human or humanized monoclonal antibodies. As used herein, “human” antibodies include antibodies having the amino acid sequence of a human immunoglobulin and include antibodies isolated from human immunoglobulin libraries or libraries of synthetic human immunoglobulin coding sequences or from mice that express antibodies from human genes.

The antibodies used in the compositions and methods of the present invention may be monospecific, bispecific, trispecific or of greater multi specificity. Multispecific antibodies may immunospecifically bind to different epitopes of Fcγ receptors, for example, to FcγRIIA and FcγRI, or, in another embodiment immunospecifically bind to both an epitope of FcγRIIB as well a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553; Todorovska et al., 2001 Journal of Immunological Methods, 248:47-66.

In one embodiment, the agent, which inhibits signaling via the FcγRIIB receptor is an antibody or an antigen-binding fragment thereof (e.g., comprising one or more complementarily determining regions (CDRs), preferably all 6 CDRs) of the antibody produced by clone 2B6 or 3H7 with ATCC accession numbers PTA-4591 and PTA-4592, respectively (e.g:, the heavy chain CDR3). In another embodiment, an antibody used in the compositions and methods of the present invention binds to the same epitope as the mouse monoclonal antibody produced from clone 2B6 or 3H7 with ATCC accession numbers PTA-4591 and PTA-4592, respectively and/or competes with the mouse monoclonal antibody produced from clone 2B6 or 3H7 with ATCC accession numbers PTA-4591 and PTA-4592, respectively as determined, e.g., in an ELISA assay or other appropriate competitive immunoassay, and also binds FcγRIIB with a greater affinity than the antibody or a fragment thereof binds FcγRIIA.

In another embodiment, the agent, which inhibits signaling via the FcγRIIB receptor is an antibody, or a fragment thereof which antagonizes signaling through the FcγRIIB receptor. In one embodiment, the antibody, may enhance intracellular calcium influx, or alter the activity of one or more downstream signaling molecules in the FcγRIIB signal transduction pathway. In another embodiment, the antibody may decrease phosphorylation of FcγRIIB or SHIP recruitment, or in another embodiment, SHIP phosphorylation, or, in another embodiment, its association with Shc. In another embodiment of the invention, the antibody may enhance MAP kinase activity, or in another embodiment, enhance activation of MAP kinase family members (e.g., Erk1, Erk2, JNK, p38, etc.). In another embodiment, the antibody may inhibit tyrosine phosphorylation, or in another embodiment, the antibody may inhibit p62dok and its association with SHIP and rasGAP. In another embodiment, the antibody may enhance FcγR-mediated phagocytosis in monocytes or macrophages.

In another embodiment, the agent may prevent signaling through the FcγRIIB receptor by preventing or inhibiting FcγRIIB receptor expression. In one embodiment, the agent may promote gene silencing. In one embodiment, gene silencing is accomplished via RNA interference, where the agent is a double-stranded RNA, which directs the sequence-specific degradation of mRNA. The agent, according to this aspect of the invention, is a small interfering RNA duplex, which may range in length typically between 20-25 nucleotides (see for example US Patent Application No. 20020086356A1), and may be constructed as will be appreciated by one skilled in the art. Sequence specific duplex RNAi mediates cleavage of the corresponding mRNA, and therefore provides a useful tool for in vivo degradation of mRNA prior to translation, hence inactivation of FcγRIIB receptor expression.

In one embodiment, the RNAi comprises duplex or double-stranded RNA, or in another embodiment, includes single-stranded RNA, isolated RNA (partially purified RNA, essentially pure RNA, synthetic RNA, recombinantly produced RNA), as well as nucleotide analogs.

In another embodiment, the agent is an antisense molecule. Antisense molecules are single-stranded nucleic acid, typically RNA, having a complementary base sequence to the base sequence of a messenger RNA (mRNA)s, whose expression is undesirable. The antisense molecule may be delivered exogenously or by introducing into the cell a vector capable of directing transcription of an antisense RNA molecule, as will be known to one skilled in the art.

Functional RNA molecules can comprise antisense oligonucleotide sequences, ribozymes comprising the antisense oligonucleotide described herein and a ribozyme sequence fused thereto. Such a ribozyme is readily synthesizable using solid phase oligonucleotide synthesis.

Ribozymes may be used for the sequence-specific inhibition of gene expression by the cleavage of mRNAs encoding proteins of interest [Welch et al., “Expression of ribozymes in gene transfer systems to modulate target RNA levels.” Curr Opin Biotechnol. October 1998;9(5):486-96]. The design of ribozymes to cleave specific target RNA may be accomplished by any means known in the art [see for example, Welch et al., “Ribozyme gene therapy for hepatitis C virus infection.” Clin Diagn Virol. Jul. 15, 1998;10(2-3):163-71.].

In another embodiment, a variety of gene knockout methods are known in the art, and may be another means of silencing expression of the FcγRIIB gene. It is to be understood that any means of silencing gene expression of, or inhibiting protein expression of, or signaling through the FcγRIIB receptor, is to be considered as part of this invention, and agents affecting the same may be utilized in the compositions and for the methods of this invention.

The composition and methods of this invention make use of an agent which inhibits signaling via the FcγRIIB receptor, as described hereinabove, and an agent, which stimulates or enhances signaling via an FcγRI receptor, an FcγRIIa receptor, an FcγRIII receptor, or a combination thereof.

In one embodiment, the agent, which stimulates or enhances signaling via an FcγRI receptor, an FcγRIIa receptor, an FcγRIII receptor, or a combination thereof is an antibody, which stimulates or enhances signaling via the FcγRI receptor, an FcγRIIa receptor, an FcγRIII receptor, or a combination thereof. In one embodiment stimulating signaling via the respective FcγR may include increased intracellular calcium influx, cell cycle progression, or activity of one or more downstream signaling molecules in the FcγR signal transduction pathway, in the cell expressing the respective FcγR. In another embodiment, enhanced phosphorylation of the FcγR may occur or SHIP recruitment. In a further embodiment of the invention, SHIP association with Shc, or inhibition of the activation of MAP kinase family members (e.g., Erk1, Erk2, JNK, p38, etc.) may occur. In another embodiment, enhanced tyrosine phosphorylation of p62dok and its association with SHIP and rasGAP. In another embodiment, the agonistic antibodies of the invention inhibit FcγR-mediated phagocytosis in monocytes or macrophages.

In one embodiment, the antibodies in compositions of this invention, or used in the methods of this invention are monoclonal, or in another embodiment, polyclonal. In one embodiment, the antibodies may be functional fragments, which engage the FcγR, as described herein.

In one embodiment, the antibodies in compositions of this invention, or used in the methods of this invention bind a human FcγR (i.e., FcγRI, FcγRIIA or FcγRIII). In one embodiment, humanized anti-FcγR monoclonal antibodies may be as described in PCT application WO 94/10332 and U.S. Pat. No. 4,954,617, the teachings of which are fully incorporated herein by reference.

The antibodies used in the compositions and methods of the present invention may be monospecific, bispecific, trispecific or of greater multi-specificity. Multispecific antibodies may immunospecifically bind to different epitopes of FcγRI, FcγRIIA, FcγRIII, or combination thereof or immunospecifically bind to both an epitope of the FcγR as well a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601.819; and Kostelny et al., 1992, J. Immunol. 148:1547-1553; Todorovska et al., 2001 Journal of Immunological Methods, 248:47-66.

In one embodiment, the agent, which stimulates or enhances signaling via an FcγRI receptor, an FcγRIIa receptor, an FcγRIII receptor, or combination thereof, is an immune complex.

In one embodiment, the immune complex comprises antigen and antibody molecules, or functional fragments thereof, as described hereinabove. In another embodiment, the immune complex may further comprise a complement protein. In one embodiment, these complexes may be somewhat insoluble, and in one embodiment, are deposited at various sites in tissue of a subject. In another embodiment, the complexes may be soluble, and may circulate in blood, over a course of time. In one embodiment, the immune complex is formed in situ at tissue sites and may be associated with immunopathological reactions, or in another embodiment, infection.

In another embodiment, the immune complex comprises a polypeptide or peptide, or protein, which is bound to the antibody or antibody fragment, and is specifically recognized by CD4 cells, or in another embodiment, is specifically recognized by CD8 cells. In another embodiment, the protein or peptide is processed intracellularly, following uptake of the immune complex by an antigen presenting cell, and is presented to both CD4+ and CD8+ T cells, or in another embodiment, each individually. In one embodiment, cross-priming of the T cells occurs.

In another embodiment, the antigen may be any molecule recognized by the immune system of the subject as foreign. For example, the antigen may be any foreign molecule, such as a protein (including a modified protein such as a glycoprotein, a mucoprotein, etc.), a nucleic acid, a carbohydrate, a proteoglycan, a lipid, a mucin molecule, or other similar molecule, including any combination thereof. The antigen may, in another embodiment, be a cell or a part thereof, for example, a cell surface molecule. In another embodiment, the antigen may derive from an infectious virus, bacteria, fungi, or other organism (e.g., protists), or part thereof. These infectious organisms may be active, in one embodiment or inactive, in another embodiment, which may be accomplished, for example, through exposure to heat or removal of at least one protein or gene required for replication of the organism. In one embodiment, the antigenic protein or peptide is isolated, or in another embodiment, synthesized.

In another embodiment, a library of peptides that span an antigenic protein is used in this invention. In one embodiment, the peptides are about 15 amino acids in length, and may, in another embodiment, be staggered every 4 amino acids along the length of the antigenic protein. In another embodiment, the antigens are obtained by recombining two or more forms of a nucleic acid that encode a polypeptide of the antigen, for example, as derived from a pathogenic agent, or antigen involved in another disease or condition. These recombination methods, referred to in one embodiment, as “DNA shuffling”, use as substrates forms of the nucleic acid that differ from each other in two or more nucleotides, so a library of recombinant nucleic acids results. The library is then screened to identify at least one optimized recombinant nucleic acid that encodes an optimized recombinant antigen that has improved ability to induce an immune response to the pathogenic agent or other condition. The resulting recombinant antigens often are chimeric in that they are recognized by antibodies (Abs) reacting against multiple pathogen strains, and generally can also elicit broad-spectrum immune responses.

In other embodiments, the different forms of the nucleic acids that encode antigenic polypeptides are obtained from members of a family of related pathogenic agents. This scheme of performing DNA shuffling using nucleic acids from related organisms, known as “family shuffling,” is described in Crameri et al. ((1998) Nature 391: 288-291). Polypeptides of different strains and serotypes of pathogens generally vary between 60-98%, which will allow for efficient family DNA shuffling. Therefore, family DNA shuffling provides an effective approach to generate multivalent, crossprotective antigens. The recombinant proteins are then produced, by methods well known to those skilled in the art, and then used in the compositions and methods of this invention.

In another embodiment, the antigen is derived from a neoplastic cell, or preneoplastic cell.

In one embodiment, the composition comprises an FcγRI, FcγRIIA, FcγRIII antibody or functional fragment thereof, bound to a cell. In one embodiment, the cell is a pathogen, or in another embodiment, the cell is neoplastic. In another embodiment, the antibody or functional fragment thereof is bound to a virus. In another embodiment, the cell is infected.

In other embodiments, the antibodies used in the compositions and methods of the invention are multi-specific with specificities for FcγRIIB, or in another embodiment, Fc receptors, e.g., FcγRI, FcγRIII, etc. and for a cancer antigen or any other cell surface marker specific for the cell of interest, or antigenic protein, or peptide, as described hereinabove.

Bispecific molecules, (e.g., heteroantibodies) comprising an anti-Fc receptor portion and an anti-target portion have been formulated and used therapeutically, e.g., for treating cancer (e.g. breast or ovarian) or pathogenic infections (e.g., HIV) (See, e.g., International Patent Application Publication No. WO 91/05871 entitled Bispecific Heteroantibodies With Dual Effector Functions; and International Patent Application Publication No. WO 91/00360 entitled Bispecific Reagents for AIDS Therapy). In addition, bispecific molecules, which recognize antigens and antigen presenting cells can be administered to a subject to stimulate an immune response (See, e.g., International Patent Application Publication No. WO 92/05793 entitled Targeted Immunostimulation With Bispecific Reagents).

Methods for preparing bi- or multivalent antibodies are for example described in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858. Binding of the single chain molecules to their specific targets can be confirmed by bispecific ELISA as will be known to one skilled in the art.

In one embodiment, the composition comprising the agents as described further comprises an inflammatory cytokine. Blockade of the FcγRII receptor, as exemplified herein, led to upregulation of DC maturation markers while the addition of inflammatory cytokines resulted in a further phenotypic maturation of the antigen presenting cell (APC) for example, with further upregulation of CD83.

In one embodiment, the inflammatory cytokine is interleukin-1β, interleukin-6, tumor necrosis factor-α or prostaglandin E2.

In another embodiment, the composition may comprise additional cytokines or growth factors, which stimulate immune responses or enhance immune responses. For example, and in one embodiment, CSF-1 may be added. In another embodiment, the composition may comprise additional therapeutic molecules, such as, for example, antibiotics, or anti cancer compounds, such as, for example, angiogenesis inhibitors.

In another embodiment, the composition may further comprise an adjuvant, such as, for example, technic acids from gram negative bacteria, such as LTA, RTA, GTA, and their synthetic counterparts, hemocyanins and hemoerythrins, such as KLH, chitin or chitosan. In another embodiment, the adjuvant may comprise muramyl dipeptide (MDP) and tripeptide peptidoglycans and their derivatives, such as threonyl-NDP, fatty acid derivatives, such as MTPPE, and the derivatives described in U.S. Pat. No. 4,950,645, incorporated herein by reference. BCG, BCG-cell wall skeleton (CWS) and trehalose monomycolate and dimycolate (U.S. Pat. Nos. 4,579,945 and 4,520,019, each incorporated herein by reference) may also be used as adjuvants in the invention, either singly or in combinations of two or three agents, or in combination with monophosphoryl lipid A (MPL) (see for example as described by Johnson et al. (1990), Grabarek et al. (1990), Baker et al. (1992; 1994); Tanamoto et al. (1994a;b; 1995); Brade et al. (1993) and U.S. Pat. No. 4,987,237). Amphipathic and surface active agents, such as QS21, and nonionic block copolymer surfactant form yet another group of preferred adjuvants. Although useful in all aspects of the invention, these adjuvants may find particular utility in compositions for use in generating or enhancing the immune response against intracellular antigens, including intracellular tumor antigens.

In another embodiment, the compositions of the invention may include bulk drug compositions useful in the manufacture of pharmaceutical compositions (e.g., impure or non-sterile compositions) and pharmaceutical compositions (i.e., compositions that are suitable for administration to a subject or patient), which can be used in the preparation of unit dosage forms. Such compositions comprise a prophylactically or therapeutically effective amount of a prophylactic and/or therapeutic agent disclosed herein or a combination of those agents and a pharmaceutically acceptable carrier.

In one embodiment, the composition comprises a therapeutically effective amount of an antibody or a fragment thereof that binds FcγRIIB with a greater affinity than the antibody or a fragment thereof binds FcγRIIA, and an agent which enhances signaling via the FcγRI, FcγRIIA, FcγRIII, or a combination thereof, and a pharmaceutically acceptable carrier.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers, in another embodiment, to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is another carrier, which, in another embodiment, is used when the pharmaceutical composition is administered intravenously. Saline solutions and aqueous dextrose and glycerol solutions can also be employed as liquid carriers, in other embodiments, including injectable solutions. Suitable pharmaceutical excipients may include, in other embodiments, starch, glucose, lactose; sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The compositions of the invention can be formulated as neutral or salt forms. Pharmaceutically acceptable salts include, but are not limited to those formed with anions such as those derived from hydrochloric, phosphoric, acetic, oxalic, tartaric acids, etc., and those formed with captions such as those derived from sodium, potassium, ammonium, calcium, ferric hydroxides, isopropylamine, triethylamine, 2-ethylamino ethanol, histidine, procaine, etc.

In another embodiment, this invention provides a method for producing an isolated, differentiated dendritic cell population, comprising contacting an immature dendritic cell with an agent which inhibits signaling via the FcγRIIB receptor and an agent which stimulates or enhances signaling via an FcγRI receptor, an FcγRIIa receptor, an FcγRIII receptor, or a combination thereof and isolating the dendritic cell, whereby the isolated dendritic cell exhibits a more differentiated phenotype than the immature dendritic cell.

In one embodiment, the term “contacting a target cell” refers herein to both direct and indirect exposure of cell to the indicated item. In one embodiment, contact of a cell with antigenic peptide, protein, cytokine, growth factor, other cell, agents of this invention, or combination thereof, is direct or indirect. In one embodiment, contacting a cell may comprise direct injection of the cell through any means well known in the art, such as microinjection. It is also envisaged, in another embodiment, that supply to the cell is indirect, such as via provision in a culture medium that surrounds the cell, or administration to a subject, via any route well known in the art, and as described hereinbelow.

Blockade of FcγRIIB, in the presence of stimulation of the other FcγR is associated not only with surface remodeling (such as upregulation of CD80/86 costimulatory molecules) associated with DC maturation.

In one embodiment, the term “dendritic cell” (DC) refers to antigen-presenting cells, which are capable of presenting antigen to T cells, in the context of MHC. In one embodiment, the dendritic cells utilized in the methods of this invention may be of any of several DC subsets, which differentiate from, in one embodiment, lymphoid or, in another embodiment, myeloid bone marrow progenitors. In one embodiment, DC development may be stimulated via the use of granulocyte-macrophage colony-stimulating-factor (GM-CSF), or in another embodiment, interleukin (IL)-3, which may, in another embodiment, enhance DC survival.

In another embodiment, DCs for use in the methods of this invention may be generated from proliferating progenitors isolated from bone marrow, as is known in the art. In another embodiment, DCs may be isolated from CD34+ progenitors as described by Caux and Banchereau (Nature 360: 258-61 1992), or from monocytes, as described by Romani et al, J. Exp. Med. 180: 83-93 1994 or Bender et al, J. Immunol. Methods, 196: 121-135, 1996. In another embodiment, the DCs are isolated from blood, as described for example, in O'Doherty et al, J. Exp. Med. 178: 1067-1078 1993 and Immunology 82: 487-493 1994, all methods of which are incorporated fully herewith by reference.

In one embodiment, the DCs utilized in the methods of this invention may express myeloid markers, such as, for example, CD11c or, in another embodiment, an IL-3 receptor-α (IL-3Rα) chain (CD 123). In another embodiment, the DCs may produce type I interferons (IFNs). In one embodiment, the DCs utilized in the methods of this invention express costimulatory molecules. In another embodiment, the DCs utilized in the methods of this invention may express additional adhesion molecules, which may, in one embodiment, serve as additional costimulatory molecules, or in another embodiment, serve to target the DCs to particular sites in vivo, when delivered via the methods of this invention, as described further hereinbelow.

In one embodiment, the DCs may be obtained from in vivo sources, such as, for example, most solid tissues in the body, peripheral blood, lymph nodes, gut associated lymphoid tissue, spleen, thymus, skin, sites of immunologic lesions, e.g. synovial fluid, pancreas, cerebrospinal fluid, tumor samples, granulomatous tissue, or any other source where such cells may be obtained. In one embodiment, the dendritic cells are obtained from human sources, which may be, in another embodiment, from human fetal, neonatal, child, or adult sources. In another embodiment, the dendritic cells used in the methods of this invention may be obtained from animal sources, such as, for example, porcine or simian, or any other animal of interest. In another embodiment, dendritic cells used in the methods of this invention may be obtained from subjects that are normal, or in another embodiment, diseased, or in another embodiment, susceptible to a disease of interest.

Dendritic cell separation may be accomplished, in another embodiment, via any separation methods as will be appreciated by one skilled in the art, and as described in part, herein. In one embodiment, positive and/or negative affinity based selections are conducted. In one embodiment, positive selection is based on CD86 expression, and negative selection is based on GR1 expression.

In another embodiment, the dendritic cells used in the methods of this invention may be generated in vitro by culturing monocytes in presence of GM-CSF and IL-4.

In one embodiment, the dendritic cells used in the methods of this invention may express CD83, an endocytic receptor to increase uptake of the antigen such as DEC-205/CD205 in one embodiment, or DC-LAMP (CD208) cell surface markers, or, in another embodiment, varying levels of the antigen presenting MHC class I and II products, or in another embodiment, accessory (adhesion and co-stimulatory) molecules including CD40, CD54, CD58 or CD86, or any combination thereof. In another embodiment, the dendritic cells may express varying levels of CD115, CD14 or CD68.

In one embodiment, mature dendritic cells are obtained by the methods of this invention. In one embodiment, the term “mature dendritic cells” refers to a population of dendritic cells with diminished CD115, CD14 or CD68 expression, or in another embodiment, a population of cells with enhanced p55, CD40, CD83, CD80 or CD86 expression, or a combination thereof. In another embodiment, mature dendritic cells obtained by the methods of this invention are characterized by CD80^{high} expression, CD83^{high} expression, CD86^{high} expression, increased MHC class II expression, increased IL-12 production or a combination thereof.

In one embodiment, the maturation status of the dendritic cell may be confirmed, for example, by detecting either one or more of 1) an increase expression of one or more of p55, CD83, CD40 or CD86 antigens; 2) loss of CD115, CD14, CD32 or CD68 antigen; by methods well known in the art, such as, for example, immunohistochemistry, FACS analysis, and others.

In one embodiment, the antigen, or in another embodiment, the immune complex is delivered to dendritic cells in vivo, and in another embodiment, in the steady state. Antigen delivery in the steady state can be accomplished, in one embodiment, as described (Bonifaz, et al. (2002) Journal of Experimental Medicine 196: 1627-1638; Manavalan et al. (2003) Transpl Immunol. 11: 245-58).

In another embodiment, the dendritic cell is contacted with the antigen, or in another embodiment, immune complex (IC) in vitro.

Dendritic cell maturation may be accompanied by, in other embodiments, enhanced antigen presentation. In one embodiment, enhanced presentation of the antigen through MHC class I, or in another embodiment, through MHC class II, or in another embodiment, both, may be accomplished, as a result of the methods of this invention. In another embodiment, use of an agent, which stimulates or enhances signaling via a combination of FcgRI receptor, FcgRIIa receptor, and FcgRIII receptor, when the agent is an immune complex, may, when presented, result in greater diversity in terms of the T cell repertoire, activated thereby, upon presentation.

Methods for priming dendritic cells with antigen are well known to one skilled in the art, and may be effected, as described for example Hsu et al., Nature Med. 2:52-58 (1996); or Steinman et al. International application PCT/US93/03141. Antigens may, in one embodiment, be chosen for a particular application, or, in another embodiment, in accordance with the methods of this invention, as described further hereinbelow, and may be associated, in other embodiments, with fungal, bacterial, parasitic, viral, tumor, or other diseases.

In one embodiment, the methods for obtaining mature dendritic cells include upregulation of costimulatory molecules on the dendritic cells, including the B7 and CD40 family of proteins. In one embodiment, such upregulation provides for enhanced stimulation of T cell proliferation and activation, and in another embodiment, prevents T cell anergy. In another embodiment, a mature dendritic cell obtained by the methods of this invention is to be considered as part of this invention.

In another embodiment, this invention provides a method for stimulating or enhancing an immune response in a subject, comprising the steps of contacting an antigen presenting cell with an agent which inhibits signaling via the FcγRIIB receptor and an agent which stimulates or enhances signaling via an FcγRI receptor, an FcγRIIa receptor, an FcγRIII receptor, or a combination thereof, whereby the antigen presenting cell contacts a T lymphocyte and the T lymphocyte stimulates or enhances an immune response in the subject, thereby being a method for stimulating or enhancing an immune response in a subject.

Blockade of FcγRIIB, in the presence of stimulation of the other FcγR is associated not only with surface remodeling (such as upregulation of CD80/86 costimulatory molecules) associated with DC maturation, but also induction of IL-12p70, which facilitates activation of T cell immunity and, in some embodiments, polarization of the response to that of a T helper-1 phenotype.

It is to be understood that any embodiment described herein, of the agent which inhibits signaling via the FcγRIIB receptor, the agent which stimulates or enhances signaling via an FcγRI receptor, an FcγRIIa receptor, an FcγRIII receptor, or a combination thereof, or compositions of this invention, are equally applicable to the methods of this invention and represent embodiments thereof. Similarly, any embodiment described herein, of the cells, immune complexes or antigens, are equally applicable to the methods of this invention and represent embodiments thereof.

In one embodiment, the agent, which stimulates or enhances signaling via an FcγRI receptor, an FcγRIIa receptor, an FcγRIII receptor, or combination thereof, is an immune complex, as described hereinabove, or in another embodiment, an antibody or antibody fragment, comprising an Fc portion which binds to said FcγRI receptor, an FcγRIIa receptor, an FcγRIII receptor, or combination thereof.

In one embodiment, an antigenic peptide or protein is contacted with antigen presenting cells, which in one embodiment, are dendritic cells, prior to contact of the dendritic cells with T cells. In one embodiment, contact of the APC's with the antigen may precede, coincide or follow contacting the APCs with the agents of this invention, as described hereinabove.

In one embodiment, soluble peptide or protein antigens are used at a concentration of between 10 pM to about 10 μM. In one embodiment, 30-100 ng ml⁻¹ is used. The APCs are, in one embodiment, contacted with the antigen for a sufficient time to allow for uptake and presentation, prior to, or in another embodiment, concurrent with contact with T cells. In another embodiment, the antigenic peptide or protein is administered to the subject, and, in another embodiment, is targeted to the APC, wherein uptake occurs in vivo, for methods as described hereinbelow.

Antigenic protein or peptide uptake and processing, in one embodiment, can occur within 24 hours, or in another embodiment, longer periods of time may be necessary, such as, for example, up to and including 4 days or, in another embodiment, shorter periods of time may be necessary, such as, for example, about 1-2 hour periods.

The enhanced immune response obtained via the methods of this invention involves T lymphocytes. The term “T lymphocytes” or “T cells” are synonymous, and refer to a subset of lymphocytes which participate in the generation of immune responses.

In one embodiment, the T cells of this invention may be obtained from in vivo sources, such as, for example, peripheral blood, leukopheresis blood product, apheresis blood product, peripheral lymph nodes, gut associated lymphoid tissue, spleen, thymus, cord blood, mesenteric lymph nodes, liver, sites of immunologic lesions, e.g. synovial fluid, pancreas, cerebrospinal fluid, tumor samples, granulomatous tissue, or any other source where such cells may be obtained. In one embodiment, the T cells are obtained from human sources, which may be, in another embodiment, from human fetal, neonatal, child, or adult sources. In another embodiment, the T cells of this invention may be obtained from animal sources, such as, for example, porcine or simian, or any other animal of interest. In another embodiment, the T cells of this invention may be obtained from subjects that are normal, or in another embodiment, diseased, or in another embodiment, susceptible to a disease of interest.

In one embodiment, the T cells and/or dendritic cells, as described further hereinbelow, of this invention are isolated from tissue, and, in another embodiment, an appropriate solution may be used for dispersion or suspension, toward this end. In another embodiment, T cells and/or dendritic cells may be cultured in solution.

Such a solution may be, in another embodiment, a balanced salt solution, such as normal saline, PBS, or Hank's balanced salt solution, or others, each of which represents another embodiment of this invention. The solution may be supplemented, in other embodiment, with fetal calf serum, bovine serum albumin (BSA), normal goat serum, or other naturally occurring factors, and, in another embodiment, may be supplied in conjunction with an acceptable buffer. The buffer may be, in other embodiments, HEPES, phosphate buffers, lactate buffers, or the like, as will be known to one skilled in the art.

In another embodiment, the solution in which the T cells or dendritic cells of this invention may be placed is in medium is which is serum-free, which may be, in another embodiment, commercially available, such as, for example, animal protein-free base media such as X-VIVO 10™ or X-VIVO 15™ (BioWittaker, Walkersville, Md.), Hematopoietic Stem Cell-SFM media (GibcoBRL, Grand Island, N.Y.) or any formulation which promotes or sustains cell viability. Serum-free media used, may, in another emodiment, be as those described in the following patent documents: WO 95/00632; U.S. Pat. No. 5,405,772; PCT US94/09622. The serum-free base medium may, in another embodiment, contain clinical grade bovine serum albumin, which may be, in another embodiment, at a concentration of about 0.5-5%, or, in another embodiment, about 1.0% (w/v). Clinical grade albumin derived from human serum, such as Buminate® (Baxter Hyland, Glendale, Calif.), may be used, in another embodiment.

In another embodiment, the T cells and/or dendritic cells may be separated via affinity-based separation methods. Techniques for affinity separation may include, in other embodiments, magnetic separation, using antibody-coated magnetic beads, affinity chromatography, cytotoxic agents joined to a monoclonal antibody or use in conjunction with a monoclonal antibody, for example, complement and cytotoxins, and “panning” with an antibody attached to a solid matrix, such as a plate, or any other convenient technique. In other embodiment, separation techniques may also include the use of fluorescence activated cell sorters, which can have varying degrees of sophistication, such as multiple color channels, low angle and obtuse light scattering detecting channels, impedance channels, etc. It is to be understood that any technique, which enables separation of the T cells or dendritic cells from a mixed source of cells, may be employed and is to be considered as part of this invention.

In another embodiment, the affinity reagents employed in the separation methods may be specific receptors or ligands for the cell surface molecules indicated hereinabove. In other embodiments, for example for T cell separations, peptide-MHC antigen and T cell receptor pairs may be used; peptide ligands and receptor; effector and receptor molecules, or others. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well known to those skilled in the art.

In another embodiment, any of the antibodies utilized herein may be conjugated to a label, which may, in another embodiment, be used for separation, or in another embodiment, for visualization of the target to which the antibody is bound. Labels may include, in other embodiments, magnetic beads, which allow for direct separation, biotin, which may be removed with avidin or streptavidin bound to, for example, a support, fluorochromes, which may be used with a fluorescence activated cell sorter, or the like, to allow for ease of separation, and others, as is well known in the art. Fluorochromes may include, in one embodiment, phycobiliproteins, such as, for example, phycoerythrin, allophycocyanins, fluorescein, Texas red, or combinations thereof.

In another embodiment, the agents of this invention are contacted with an antigen presenting cell, in vivo, or ex-vivo, and the agent is labeled with a detectable marker, such that, in one embodiment, homing, or in another embodiment, persistence of the labeled APC may be followed as a function of time.

In one embodiment, the staining intensity of the cells can be monitored by flow cytometry, where lasers detect the quantitative levels of fluorochrome (which is proportional to the amount of cell surface antigen bound by the antibodies). Flow cytometry, or FACS, can also be used, in another embodiment, to separate cell populations based on the intensity of antibody staining, as well as other parameters such as cell size and light scatter.

In another embodiment, the culture containing the T cells and/or APCs of this invention may contain cytokines or growth factors to which the cells are responsive. In one embodiment, the cytokines or growth factors promote survival, growth, function, or a combination thereof of the T and/or dendritic cells. Cytokines and growth factors may include, in other embodiment, polypeptides and non-polypeptide factors. In one embodiment, the cytokines may comprise interleukins.

In one embodiment, the T cell populations, once contacted with the dendritic cells, according to the methods of this invention, are antigen specific. In one embodiment, the term “antigen specific” refers to a property of the population such that supply of a particular antigen, or in another embodiment, a fragment of the antigen, results, in one embodiment, in specific T cell proliferation, when presented the antigen, in the context of MHC. In another embodiment, supply of the antigen or fragment thereof, results in T cell production of interleukin 2, or in another embodiment, interferon-γ, or in another embodiment, enhanced expression of the T cell receptor (TCR) on its surface, or in another embodiment, T cell function, such as, for example, cytolysis.

In one embodiment, the T cell population expresses a monoclonal T cell receptor. In another embodiment, the T cell population expresses polyclonal T cell receptors.

In one embodiment, the T cells will be of one or more specificities, and may include, in another embodiment, those that recognize a mixture of antigens derived from a single antigenic source, such as, for example, in infection, where recognition of multiple epitopes of a given antigen may be used to expand the T cells.

In one embodiment, the T cell population stimulates or enhances an immune response to a particular antigen, wherein the immune response generated is beneficial to the host, such as, for example, a response directed against an antigen from a pathogen that has invaded the subject.

In one embodiment, the T cell populations secrete substances, which mediate desirable effects, such as promoting the activation of other immune cells, in one embodiment, or promote lysis in another embodiment, or apoptosis, in another embodiment. In one embodiment, the T cells of this invention mediate their effect on the immune system, without a need for direct cell contact. In one embodiment, the substances mediating these effects secreted by the T cell populations may include IL-2, interferon-γ, or a combination thereof.

In another embodiment, the T cell populations may be engineered to express substances which when secreted mediate stimulatory effects on the immune system, such as, for example, the cytokines listed hereinabove. In another embodiment, the T cell populations may be engineered to express particular adhesion molecules, or other targeting molecules, which, when the cells are provided to a subject, facilitate targeting of the T cell populations to a site of interest. For example, when T cell activity is desired to stimulate, or enhance an immune response at a mucosal surface, the T cell populations may be further engineered to express the αₑβ₇ adhesion molecule, which has been shown to play a role in mucosal homing. The cells can be engineered to express other targeting molecules, such as, for example, an antibody specific for a protein expressed at a particular site in a tissue, or, in another embodiment, expressed on a particular cell located at a site of interest, etc. Numerous methods are well known in the art for engineering the T cells and/or dendritic cells, as described herein, and may comprise the use of a vector, or naked DNA, wherein a nucleic acid coding for the targeting molecule of interest is introduced via any number of methods well described.

A nucleic acid sequence of interest may be subcloned within a particular vector, depending upon the desired method of introduction of the sequence within cells. Once the nucleic acid segment is subcloned into a particular vector it thereby becomes a recombinant vector. Polynucleotide segments encoding sequences of interest can be ligated into commercially available expression vector systems suitable for transducing/transforming mammalian cells and for directing the expression of recombinant products within the transduced cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides.

There are a number of techniques known in the art for introducing the above described recombinant vectors into cells, such as, but not limited to: direct DNA uptake techniques, and virus, plasmid, linear DNA or liposome mediated transduction, receptor-mediated uptake and magnetoporation methods employing calcium-phosphate mediated and DEAE-dextran mediated methods of introduction, electroporation, liposome-mediated transfection, direct injection, and receptor-mediated uptake (for further detail see, for example, “Methods in Enzymology” Vol. 1-317, Academic Press, Current Protocols in Molecular Biology, Ausubel F. M. et al. (eds.) Greene Publishing Associates, (1989) and in Molecular Cloning: A Laboratory Manual, 2nd Edition, Sambrook et al. Cold Spring Harbor Laboratory Press, (1989), or other standard laboratory manuals). Bombardment with nucleic acid coated particles is also envisaged.

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay. If the gene product of interest to be expressed by a cell is not readily assayable, an expression system can first be optimized using a reporter gene linked to the regulatory elements and vector to be used. The reporter gene encodes a gene product, which is easily detectable and, thus, can be used to evaluate efficacy of the system. Standard reporter genes used in the art include genes encoding β-galactosidase, chloramphenicol acetyl transferase, luciferase and human growth hormone, or any of the marker proteins listed herein.

In one embodiment, T cells may be contacted with APC's at a ratio of 1:1 to 1:10. In one embodiment, the T cells used in the methods of this invention, are autologous, or, in another embodiment, syngeneic or, in another embodiment, allogeneic, with respect to the dendritic cells, and in another embodiment, with respect to the subject.

In one embodiment, the agent, which stimulates or enhances signaling via an FcγRI receptor, an FcγRIIa receptor, an FcγRIII receptor, or combination thereof, is an immune complex, which comprises a polypeptide or peptide, bound to an antibody or antibody fragment, whose Fc portion, in another embodiment, binds to the FcγRI receptor, an FcγRIIa receptor, an FcγRIII receptor, or combination thereof

In one embodiment, the polypeptide or peptide is increasingly or preferentially expressed during disease or infection. In one embodiment, the disease is cancer.

According to this aspect of the invention, and in one embodiment, the subject being treated by the method of this invention has, has had, or is at increased risk for the disease, which in one embodiment is cancer.

FcγRIIB blockade of DCs loaded with tumor cells led to increased tumor specific T cell immunity, without the need for exogenous stimuli other than human plasma Therefore the activation status of DCs in the presence of normal human serum depends on the balance between activating and inhibitory FcγR and can be enhanced by agents that selectively inhibit signaling through FcγRIIB.

In one embodiment, this invention provides a method for treating, suppressing, or preventing cancer in a subject, the method comprising the steps of contacting an immature dendritic cell with an agent which inhibits signaling via the FcγRIIB receptor and an agent, which stimulates or enhances signaling via an FcγRI receptor, an FcγRIIa receptor, an FcγRIII receptor, or a combination thereof, whereby the dendritic cell contacts a T lymphocyte and the T lymphocyte stimulates or enhances an immune response against a cancer in the subject, thereby being a method for treating, suppressing, or preventing cancer in a subject.

Accordingly, the methods and compositions of the invention are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosafcoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions of the invention in the ovary, bladder, breast, colon, lung, skin, pancreas, or uterus. In other embodiments, sarcoma, melanoma, or leukemia is treated or prevented by the methods and compositions of the invention.

Cancers associated with altered expression of particular antigens, or with unique cancer antigens may be treated or prevented by the methods, and utilizing the compositions of this invention. In one embodiment, the agent, which inhibits FcγRIIB signaling is admistered in combination with an agent that stimulates signaling via another FcγR. In one embodiment, the latter agent is an antibody, which may bind the cancer antigen or antigen preferentially expressed in neoplasia/preneoplasia. In one embodiment, the latter antibody enhances the immune response, via enhancing antigen presentation. In one embodiment, the immune response is enhanced via cross-priming to both T helper and cytotoxic T lymphocytes. In one embodiment, the T cells thus primed, have enhanced cytokine production, which in another embodiment, activates other cells of the immune system, and in another embodiment, these cells lyse or cause apoptosis in neoplastic cells. In another embodiment, the cytotoxic T lymphocytes (CTLs) generated lyse neoplastic cells.

In one embodiment, cancers associated with the following cancer antigen may be treated or prevented by the methods and compositions of the invention. K S 1/4 pan-carcinoma antigen (Perez and Walker, 1990, J. Immunol. 142:32-37; Bumal, 1988, Hybridoma 7(4):407-415), ovarian carcinoma antigen (CA125) (Yu et al., 1991, Cancer Res. 51(2):48-475), prostatic acid phosphate (Tailor et al., 1990, Nucl. Acids Res. 18(1):4928), prostate specific antigen (Henttu and Vihko, 1989, Biochem. Biophys. Res. Comm. 10(2):903-910; Israeli et al., 1993, Cancer Res. 53:227-230), melanoma-associated antigen p97 (Estin et al., 1989, J. Natl. Cancer Instit. 81 (6):445-44), melanoma antigen gp75 (Vijayasardahl et al., 1990, J. Exp. Med. 171(4):1375-1380), high molecular weight melanoma antigen (HMW-MAA) (Natali et al., 1987, Cancer 59:55-3; Mittelman et al., 1990, J. Clin. Invest. 86:2136-2144)), prostate specific membrane antigen, carcinoembryonic antigen (CEA) (Foon et al., 1994, Proc. Am. Soc. Clin. Oncol. 13:294), polymorphic epithelial mucin antigen, human milk fat globule antigen, Colorectal tumor-associated antigens such as: CEA, TAG-72 (Yokata et al., 1992, Cancer Res. 52:3402-3408), CO17-1A (Ragnhammar et al., 1993, Int. J. Cancer 53:751-758); GICA 19-9 (Herlyn et al., 1982, J. Clin. Immunol. 2:135), CTA-1 and LEA, Burkitt's lymphoma antigen-38.13, CD19 (Ghetie et al., 1994, Blood 83:1329-1336), human B-lymphoma antigen-CD20 (Reffet al., 1994, Blood 83:435-445), CD33 (Sgouros et al., 1993, J. Nucl. Med. 34:422430), melanoma specific antigens such as ganglioside GD2 (Saleh et al., 1993, J. Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53:5244-5250), tumor-specific transplantation type of cell-surface antigen (TSTA) such as virally-induced tumor antigens including T-antigen DNA tumor viruses and envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46:3917-3923), antigens of fibrosarcoma, human leukemia T cell antigen-Gp37 (Bhattacharya-Chattejee et al., 1988, J. of Immun. 141:1398-1403), neoglycoprotein, sphingolipids, breast cancer antigen such as EGFR (Epidermal growth factor receptor), HER2 antigen (p185HER2), polymorphic epithelial mucin (PEM) (Hilkens et al., 1992, Trends in Bio. Chem. Sci. 17:359), malignant human lymphocyte antigen-APO-1 (Bernhard et al., 1989, Science 245:301-304), differentiation antigen (Feizi, 1985, Nature 314:53-57) such as I antigen found in fetal erthrocytes and primary endoderm, I(Ma) found in gastric adencarcinomas, M18 and M39 found in breast epithelium, SSEA-1 found in myeloid cells, VEP8, VEP9, Myl, VIM-D5, and D156-22 found in colorectal cancer, TRA-1-85 (blood group H), C14 found in colonic adenocarcinoma, F3 found in lung adenocarcinoma, AH6 found in gastric cancer, Y hapten, Ley found in embryonal carcinoma cells, TL5 (blood group A), EGF receptor found in A431 cells, E1 series (blood group B) found in pancreatic cancer, FC10.2 found in embryonal carcinoma cells, gastric adenocarcinoma, CO-514 (blood group Lea) found in adenocarcinoma, NS-10 found in adenocarcinomas, CO-43 (blood group Leb), G49, EGF receptor, (blood group ALeb/Ley) found in colonic adenocarcinoma, 19.9 found in colon cancer, gastric cancer mucins, T5A7 found in myeloid cells, R24 found in melanoma, 4.2, GD3, D1.1, OFA-1, GM2, OFA-2, GD2, M1:22:25:8 found in embryonal carcinoma cells and SSEA-3, SSEA-4 found in 4-8-cell stage embryos. In another embodiment, the antigen is a T cell receptor derived peptide from a cutaneous T cell lymphoma (see Edelson, 1998, The Cancer Journal 4:62).

In another embodiment, the antigenic peptide or protein is derived from HER2/neu or chorio-embryonic antigen (CEA) for suppression/inhibition of cancers of the breast, ovary, pancreas, colon, prostate, and lung, which express these antigens. Similarly, mucin-type antigens such as MUC-1 can be used against various carcinomas; the MAGE, BAGE, and Mart-1 antigens can be used against melanomas. In one embodiment, the methods may be tailored to a specific cancer patient, such that the choice of antigenic peptide or protein is based on which antigen(s) are expressed in the patient's cancer cells, which may be predetermined by, in other embodiments, surgical biopsy or blood cell sample followed by immunohistochemistry.

In another embodiment, the polypeptide or peptide is increasingly or preferentially expressed during infection.

In one embodiment, the methods and compositions of the invention can be used to enhance a specific immune response directed against an antigen associated with an infection. The antigen may be derived from infectious agents, diseased or abnormal cells such as, but not limited to, bacteria (e.g., gram positive bacteria, gram negative bacteria, aerobic bacteria, Spirochetes, Mycobacteria, Rickettsias, Chlamydias, etc.), parasites, fungi (e.g., Candida albicans, Aspergillus, etc.) or viruses (e.g., DNA viruses, RNA viruses, etc.).

Examples of infectious virus to which stimulation of an immune response according to the methods of this invention may be applicable include: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g. influenza viruses); Bungaviridae (e.g., Hantaan viruses, bunga viruses, phleboviruses and Nairo viruses); Arena viridae (hemorrhagic fever viruses); Reoviridae (erg., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, Epstein Barr virus, cytomegalovirus (CMV), herpes viruses'); Poxviridae (variola viruses, vaccinia viruses, pox viruses); and Iridoviridae (e.g. African swine fever virus); and unclassified viruses (e.g., the etiological agents of Spongiform encephalopathies, the agent of delta hepatities (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Examples of infectious bacteria to which stimulation of an immune response according to the methods of this invention may be applicable include: Helicobacter pylori, Borellia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Chlamidia sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Actinomyces israelli and Francisella tularensis.

Examples of infectious fungi to which stimulation of an immune response according to the methods of this invention may be applicable include: Cryptococcus neoformans, Histoplasma capsulatum, Coccidioides immitis, Blastomyces dermatitidis, Chlamydia trachomatis, Candida albicans. Other infectious organisms (i.e., protists) include: Plasmodium sp., Leishmania sp., Schistosoma sp. and Toxoplasma sp.

In one embodiment, the methods of enancing an immune response in a subject, where the response is directed against a neoplastic cell or infection, involves the use of dendritic cells and/or T lymphocytes isolated from a subject afflicted with cancer, or in another embodiment, wherein the subject has precancerous precursors for cancer, or in another embodiment, wherein the subject is at increased risk for cancer, or in another embodiment, wherein the subject is infected, or in another embodiment, wherein the subject is at increased risk of infection, from a particular pathogen.

In one embodiment, the invention provides methods and compositions for stimulating T cells, and preventing T cell anergy. In one embodiment, the method and compositions of this invention prvent anergy via upregulation of costimulatory molecules, such as, for example B7, CD40 and/or ICAM-1 on the APC surface.

FcγRIIB blockade of DCs resulted in enhanced production of IL-12, when the DCs were further stimulated through other FcγR. IL-12 is a potent stimulator of immune responses, and of T cells in particular, thus, in another embodiment, the methods and compositions of this invention may be understood to be applicable to stimulating immune responses in patients with undesirable T cell anergy, such as occurs, for example, in tuberculosis infection.

Moreover, IL-12 is known to participate in stimulating the so-called Th1 responses. In one embodiment, the methods and compositions of this invention may be involved in modulating immune responses.

In one embodiment, the term “modulating” refers to stimulating, enhancing or altering the immune response. In one embodiment, the term “enhancing an immune response” refers to any improvement in an immune response that has already been mounted by a subject. In another embodiment, the term “stimulating an immune response” refers to the initiation of an immune response against an antigen of interest in a subject in which an immune response against the antigen of interest has not already been initiated. It is to be understood that reference to modulation of the immune response may, in another embodiment, involve both the humoral and cell-mediated arms of the immune system, which is accompanied by the presence of Th2 and Th1 T helper cells, respectively, or in another embodiment, each arm individually. For further discussion of immune responses, see, e.g., Abbas et al. Cellular and Molecular Immunology, 3rd Ed., W. B. Saunders Co., Philadelphia, Pa. (1997).

In another embodiment, modulation of the immune response may result in the eliciting a “Th1” response, in a disease where a so-called “Th2” type response has developed, when the development of a so-called “Th1” type response is beneficial to the subject. One example would be in leprosy, where the antigen stimulates a Th1 cytokine shift, resulting in tuberculoid leprosy, as opposed to lepromatous leprosy, a much more severe form of the disease, associated with Th2 type responses.

Modulation of an immune response can be determined, in one embodiment, by measuring changes or enhancements in production of specific cytokines and/or chemokines for either or both arms of the immune system. In one embodiment, modulation of the immune response resulting in the stimulation or enhancement of the cell mediated immune response, may be reflected by an increase in interferon-γ, which can be determined by any number of means well known in the art, such as, for example, by ELISA or RIA.

In one embodiment, stimulating, enhancing or altering the immune response is associated with a change in cytokine profile. In another embodiment stimulating, enhancing or altering said immune response is associated with a change in cytokine expression. Such changes may be readily measured by any number of means well known in the art, including as described herein, ELISA, RIA, Western Blot analysis, Northern blot analysis, PCR analysis, RNase protection assays, and others.

In another embodiment, stimulating, enhancing or altering the immune response according to the methods, and using the compositions of this invention may be associated with enhanced production of reactive oxidative species, including reactive oxygen intermediates, such as peroxide production, or, in another embodiment, reactive nitrogen intermediate production, such as, for example, in enhancing nitric oxide production.

In another embodiment, the methods and compositions of this invention may be particularly applicable in subjects with a latent infection. In another embodiment, methods and compositions of this invention may be particularly applicable in subjects with an immune response, which is not protective to the subject, or in another embodiment, wherein the subject exhibits a cytokine profile that exacerbates disease.

In another embodiment, the methods for modulating immune responses in a subject of this invention may further comprise the step of administering an agent to the subject, which elicits a cytokine profile in the subject associated with protection from the pathogen. In one embodiment, the immune response prevents infection in the subject. In another embodiment, the immune response prevents latent infection in the subject.

In one embodiment, cells for administration to a subject in this invention may be provided in a composition. These compositions may, in one embodiment, be administered parenterally or intravenously. The compositions for administration may be, in one embodiment, sterile solutions, or in other embodiments, aqueous or non-aqueous, suspensions or emulsions. In one embodiment, the compositions may comprise propylene glycol, polyethylene glycol, injectable organic esters, for example ethyl oleate, or cyclodextrins. In another embodiment, compositions may also comprise wetting, emulsifying and/or dispersing agents. In another embodiment, the compositions may also comprise sterile water or any other sterile injectable medium. In another embodiment, the compositions may comprise adjuvants, which are well known to a person skilled in the art (for example, vitamin C, antioxidant agents, etc.) for some of the methods as described herein, wherein stimulation of an immune response is desired, as described further hereinbelow.

In one embodiment, the cells or compositions of this invention may be administered to a subject via injection. In one embodiment, injection may be via any means known in the art, and may include, for example, intra-lymphoidal, or subcutaneous injection.

In another embodiment, the T cells and dendritic cells for administration in this invention may express adhesion molecules for targeting to particular sites. In one embodiment, T cell and/or dendritic cells may be engineered to express desired molecules, or, in another embodiment, may be stimulated to express the same. In one embodiment, the DC cells for administration in this invention may further express chemokine receptors, in addition to adhesion molecules, and in another embodiment, expression of the same may serve to attract the DC to secondary lymphoid organs for priming. In another embodiment, targeting of DCs to these sites may be accomplished via injecting the DCs directly to secondary lympoid organs through intralymphatic or intranodal injection.

In one embodiment, the antigen presenting cells are contacted with the agent, which inhibits signaling via the FcYRIIB receptor in vivo, and the agent which stimulates or enhances signaling via an FcγRI receptor, or an FcYRIIa receptor, or an FcYRIII receptor; or T lymphocyte or combination thereof is contacted in vivo. In another embodiment, the antigen presenting cells are contacted with the agent which inhibits signaling via the FcYRIIB receptor ex vivo, and the agent which stimulates or enhances signaling via an FcγRI receptor, or an FcYRIIa receptor, or an FcYRIII receptor; or T lymphocyte or combination thereof is contacted in vivo, or ex vivo. In another embodiment, the antigen presenting cells are contacted with the agent, which inhibits signaling via the FcYRIIB receptor in vitro, and the agent which stimulates or enhances signaling via an FcγRI receptor, or an FcYRIIa receptor, or an FcYRIII receptor; or T lymphocyte or combination thereof is contacted in vivo, or, in another embodiment, ex-vivo. In another embodiment, the antigen presenting cells are contacted with the agent, which inhibits signaling via the FcYRIIB receptor in vitro, ex-vivo, or in vivo and the agent which stimulates or enhances signaling via an FcγRI receptor, or an FcYRIIa receptor, or an FcYRIII receptor, each individually is contacted in vitro, or ex-vivo, or in vivo with the dendritic cell and/or the T lymphocyte is contacted with the dendritic cell, in vitro, or in another embodiment, in vivo, or, in another embodiment, ex-vivo.

In one embodiment, the dendritic cells and/or T cells of this invention may be administered to a recipient contemporaneously with the agents of this invention, and with an immune complex or antigen. In another embodiment, the dendritic cells and/or T cells of this invention may be administered prior to the administration of the agents of this invention, and/or with an immune complex or antigen. In one embodiment, the dendritic cells and/or T cells of this invention may be administered to the recipient about 3 to 7 days before administration of the agents of this invention, and/or with an immune complex or antigen.

The dosage of the dendritic cells and/or T cells varies, in other embodiments, within wide limits and will be fitted to the individual requirements in each particular case, and may be, in another embodiment, a reflection of the weight and condition of the recipient, the number of or frequency of administrations, and other variables known to those of skill in the art. The dendritic cells and/or T cells can be administered, in other embodiments, by a route, which is suitable for the tissue, organ or cells to be treated. The dendritic cells and/or T cells of this invention may be administered systemically, i.e., parenterally, by intravenous injection or targeted to a particular tissue or organ, such as bone marrow, or lymph nodes, or an infected or neoplastic organ, or lobe of an organ. The dendritic cells and/or T cells of this invention may, in another embodiment, be administered via a subcutaneous implantation of cells.

The following non-limiting examples may help to illustrate some embodiments of the invention.

### EXAMPLES

### Materials and Methods

Generation of Dendritic Cells (DCs):

Peripheral blood monocytes (PBMCs) were obtained from leucocyte concentrates of healthy blood donors (purchased from New York Blood Center, NY) by density gradient centrifugation using Ficoll-Hypaque (Amersham Pharmacia, Biotech, Uppsala, Sweden). CD14+ cells were separated using CD14 microbeads and columns (Miltenyi Biotech) following the manufacturer's protocol and cultured in RPMI-1640 medium with L-glutamine (Mediatech, Herndon, Va.) supplemented with 1% single donor plasma and gentamycin (20 μg/ml; Bio Whittaker). For some experiments, DC cultures were performed in serum free media (AIM-V medium, Gibco). Additional DC culture media as controls in some experiments included serum free media supplemented with 1% plasma, or RPMI-1640 supplemented with Ig depleted 1% plasma. Igs were depleted from plasma using affinity chromatography on a protein G-Sepharose column (Pharmacia), and depletion verified using SDS-PAGE. GMCSF (20 ng/ml; Immunex, Seattle, Wash.) and IL-6 (12.5 ng/ml; R&D Systems, Minneapolis, Minn.) were added to the medium on days 0, 2 and 4 of culture. For some experiments, DCs were matured using an inflammatory cytokine cocktail (Jonuleit, H., et al. (1997) Eur. J. Immunol. 27, 3135-3142.) consisting of IL-1β 10 ng/ml, IL-6 1000 U/ml and TNF-α 10 ng/ml (all from R&D Systems) and PGE2, 1 mg/ml (Sigma, St. Louis, Mo.).

To isolate blood derived DCs, PBMCs were stained with a lineage antibody cocktail (Lin-1 FITC; Miltenyi Biotec) containing anti-CD3, CD14, CD16, CD19, CD20 and CD56. The lineage negative fraction was isolated using anti-FITC magnetic microbeads (as per the manufacturer's recommendations (Miltenyi Biotec), followed by fluorescence activated cell sorting. Myeloid DCs were identified as being Lin-1 negative, HLA-DR^{high}, and CD11c+ cells. Plasmacytoid DCs were identified as being Lin-1 negative, HLA-DR^{high}, and either CD123 or BDCA-2+ cells.

FcγRIIB Blocking Antibodies:

Antibodies that selectively bind and block human FcγRIIB (2B6) were obtained from Macrogenics Inc. Initial experiments utilized a mouse monoclonal antibody (clone 2B6). Additional constructs that were tested included a human-mouse chimeric antibody (ch-2B6), as well as an aglycosylated version (agly-2B6), designed to minimize binding via the Fc region of the antibody. Antibodies were generally used at a concentration of 1-25 mg/ml, with 1 mg/ml saturating the capacity of 2B6 to induce DC maturation.

Blocking Inhibitory FcγR on Human Monocyte Derived DCs:

To block inhibitory FcγR, immature DCs on day 5 of culture were treated with either anti-human FcγRIIB blocking antibody (2B6, Macrogenics), IgG1 isotype-matched control antibody (Sigma, St. Louis, Mo.), anti-CD16 receptor blocking antibody (clone 3G8 from Becton Dickinson, San Jose, Calif.) or left untreated for 3 hours at 37° C.

Phenotyping of DCs and Evaluation of Their Maturation:

Immature DCs were cultured either alone or fed on day 5 of culture with antibody coated dying tumor cells, as described (Dhodapkar, K., et al. (2002) J Exp Med 195, 125-133). Prior to culture, the DCs were either untreated or treated with FcγRIIB blocking antibody 2B6, or IgG1 isotype-matched control antibody as above. After 8 hours of culture, maturation cytokines were added to some of the DC cultures. DCs were harvested about 24 hours later, stained and subjected to flow cytometric analysis. The following antibodies were used for evaluating surface marker expression changes associated with DC maturation; CD11c-APC, CD80-PE, CD86-FITC, CD86-PE, HLA-DR-FITC (all obtained from Becton Dickinson, San Jose, Calif.). In addition immature DCs as well as DCs matured using the cytokine cocktail were tested for the presence of both the inhibitory receptor FcγRIIB (using anti-FcγRIIB receptor antibody 2B6 FITC, Macrogenics) and the activating Fc receptor FcγRIIa (clone IV.3FITC; Medarex).

Enzyme Linked Immunoassay (ELISA) to Measure the Production of IL-12p70 by DCs:

DCs cultured from purified monocytes were treated with FcγRIIB blocking antibody, or isotype control as described above. After overnight culture, supernatants were harvested and analyzed for the presence of IL-12p70 by ELISA (R&D Diagnostics), using the manufacturer's recommendations.

Myeloma Cell-Lines:

Myeloma cell lines were obtained from American Type Culture Collection (U266 cells) or provided by J. Epstein, Arkansas Cancer Research Center, Little Rock, Ark. (cag cells). Both lines were maintained in RPMI-1604 with L-glutamine, supplemented with 10% fetal bovine serum and gentamicin.

Loading of Antibody Coated Dying Tumor Cells on DCs:

U266 cells were labeled with anti-syndecan-1 antibody (1 mg/ml, B-B4; Serotec) for 30 minutes at 37° C. and then washed and irradiated to 30 Gy. The irradiated tumor cells were immediately co-cultured with the immature DCs alone (DC: tumor ratio 1:1, DCs at 0.5×10⁶ cells/ml in 200 μl 5% PHS), or DCs pre-coated with FcγR blocking antibody or isotype-matched control antibody, (4×10⁶ DCs/ml pretreated with 1 μg/ml), for 30 minutes at 37° C. Some of the DCs were matured 8 hours later using a cocktail of cytokines as above. DCs were used for T cell stimulation after overnight culture with the tumor cells with or without the addition of maturation cytokines.

Evaluation of Tumor Cell Uptake:

To evaluate phagocytosis of dying tumor cells by DCs, live tumor cells were labeled red with PHK26 (Sigma-Aldrich; St Louis, Mo.) and immature DCs were labeled green using PKH67 (Sigma-Aldrich; St Louis, Mo.) as per the manufacturer's protocol. The tumor cells were then left either uncoated or coated with anti-syndecan-1 antibody, irradiated and co-cultured with the dye labeled DCs at either 4° C. or 37° C. After 4-8 hours of co-culture, tumor uptake was determined by evaluating the double positive cells seen by flow cytometry.

Stimulation of T Cells:

CD14 negative blood mononuclear cells were used as the source of T cells. CD56 cells were depleted from the CD14-cells using CD56 microbeads (Miltenyi biotech). CD56 depleted T cells were stimulated in 24 well flat bottom plates in RPMI 1640 with L-glutamine supplemented with 5% pooled human serum. DCs were added to the T cells at a ratio of 1:10-1:30 on days 0 and 7 of culture. IL-2 25-50 U/ml (Chiron, Emeryville, Calif.) was added on day 2 and 7 of culture. Cultures were tested for the presence of tumor specific T cells 7-10 days after the last stimulation with DCs.

Evaluation of Tumor Reactive Interferon-γproducing T Cells:

The induction of tumor reactive, interferon-γ producing T cells by tumor loaded DCs was assessed using an ELISPOT assay, as described (27). For ELISPOT assay, 10⁵ T cells were co-cultured overnight with tumor cells (T: tumor cell ratio of 20:1) in ELISPOT plates pre-coated with anti-interferon-γ antibody (Mabtech, Sweden). To detect peptide specific T cells, autologous DCs were pulsed with 10 mM HLA A2 restricted peptides derived from MAGE-A3 (271-279; FLWGPRALV (SEQ ID NO: 1)), NY-ESO-1 (157-165; SLLMWITQC (SEQ ID NO: 2)), or 2.5 mM of an overlapping peptide library (15 mer peptides overlapping by 11 aa) derived from survivin. The peptides were synthesized by the proteomics resource center at Rockefeller University. The peptide pulsed DCs were washed and used as APCs in the Elispot assay, as described (Dhodapkar, K., et al. (2002) J Exp Med 195, 125-133).

### Example 1

### Expression of Both Inhibitory (FcγRIIb) and Activating Fc Receptor (FcγRIIa) on Immature and Mature Monocyte-Derived DCs

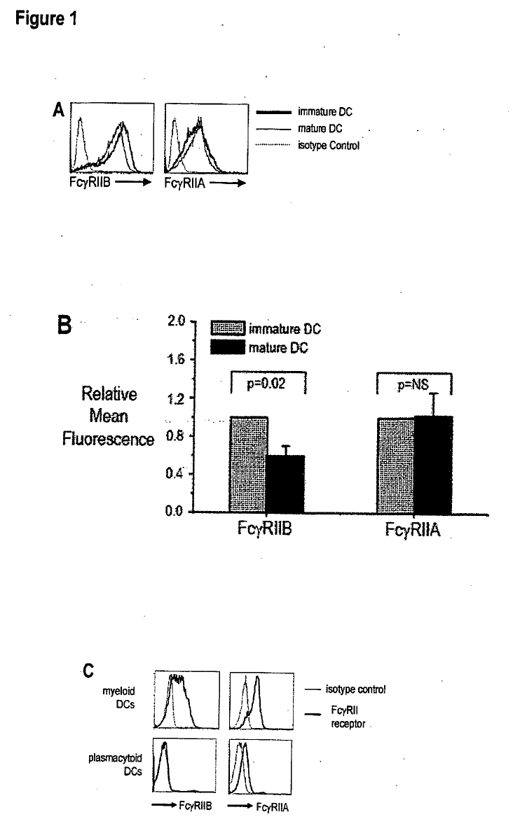

Prior studies have shown that human monocyte-derived DCs express FcγRII, and RIIII, but not FcγRI. However, these studies did not specifically examine the pattern of activating versus inhibitory Fc receptors on these DCs. The expression of both inhibitory (FcγRIIb) and activating (FcγRIIa) Fc receptors was therefore examined on pure populations of monocyte derived (CD14+) DCs in the immature stage and after maturation with the cytokine cocktail. Immature and mature DCs express both activating and inhibitory FcγRII receptors (FIG. 1A). Since differences in staining intensity of activating and inhibitory FcγR antibodies may be due to intrinsic differences in antibodies or efficiency of fluorochrome conjugation, the ratio of mean fluorescence intensity (MFI) of staining with these antibodies between immature and mature DCs was compared (FIG. 1B). In two experiments, the RIIB/RIIA ratio was higher in immature DCs and decreased after DC maturation. Thus both immature and mature human DCs express both inhibitory and activating receptors, but the relative proportion of these may change with maturation.

To confirm that the 2B6 antibody specifically binds the FcγRIIB receptor (CD32B) and not CD32A on human DCs, surface marker expression was determined in myeloid (lineage negative, HLA-DR+, CD11c+) and plasmacytoid (lineage negative, HLA-DR+, and CD123/BDCA2+) subsets of blood DCs. Plasmacytoid DCs express CD32A, but do not express the CD32B gene, and consistent with this, plasmacytoid DCs expressed CD32A but did bind the 2B6 mAb, while in contrast, myeloid DCs stained well with specific antibodies against both activating and inhibitory receptor (FIG. 1C).

### Example 2

### Blocking Inhibitory FcγRIIB on Immature DCs Leads to a Mature Cell Surface Phenotype

Serum from otherwise healthy adults can contain circulating immune complexes (up to 50-100 μg/ml), which in principle may engage FcγRs on DCs. We hypothesized that the lack of spontaneous DC maturation during culture in human plasma was due to co-engagement of activation and inhibitory receptors. To test this more directly, DCs cultured in the presence of 1% normal human plasma were pretreated with an FcγRIIB blocking (2B6) antibody and DC maturation was monitored for the upregulation of surface markers. FcγRIIB blockade was associated with up-regulation of CD83, as well as co-stimulatory molecules (CD80 and CD86), and HLA-DR. DC maturation associated with FcγRIIB blockade was seen only when the DCs were cultured in the presence of human plasma, but not in serum/plasma free media (FIG. 2A). To further characterize the effects on DC maturation, we utilized two additional constructs of the same antibody; a mouse-human chimeric antibody (2B6-ch) and an aglycosylated version (2B6-agly) designed to minimize binding via the Fc portion. Addition of 1% plasma to serum free media reconstituted the DC maturation seen after FcγRIIB blockade (FIG. 2B). Depletion of immunoglobulin (Ig) from plasma in the DC culture media using a protein G/sepharose column attenuated the upregulation of DC maturation markers (FIG. 2B). Thus, the blockade of FcγRIIB on human DCs leads to DC maturation in the presence of activating Ig ligands in the normal human serum.

### Example 3

### Blocking Inhibitory FcγRIIB on Immature DCs Leads to IL-12 Production

Maturation of DCs by certain ligands, such as ligands for toll receptors or CD40L, leads to the secretion of IL-12, which plays a major role in allowing DCs to differentiate CD4+ T cells along the T helper 1 type pathway, needed for protection against tumors and pathogens. Blockade of FcγRIIB on pure populations of monocyte derived human DCs led to the production of IL-12p70 (FIG. 3). In contrast, DCs matured using the inflammatory cytokine cocktail that is commonly used in DC vaccination trials are poor IL-12p70 producers, as noted previously, in the presence or absence of isotype control antibody. These data show that in the presence of activating ligands present in normal human sera, simple blockade of inhibitory Fcγ receptors induces DCs to secrete IL-12p70.

### Example 4

### Blocking the Inhibitory Fcγ Receptor FcγRIIB Enhances the Generation of Tumor Reactive T Cell Immunity by Tumor Loaded Human Monocyte-Derived Dcs

DCs can acquire antigen from tumor or virally infected cells, and cross-present the acquired antigens to elicit antigen specific CD8+ T cells. In prior studies, the coating of tumor cells with anti-tumor monoclonal antibodies prior to uptake by human DCs has been shown to lead to enhanced cross-presentation. This process depends on the engagement of FcγRs on DCs. However, the uptake of antibody-coated cells likely causes simultaneous engagement of both activating and inhibitory receptors. Therefore blockade of the inhibitory FcγR was evaluated for its ability to alter DC maturation, in the context of DC generation of T cell immunity.

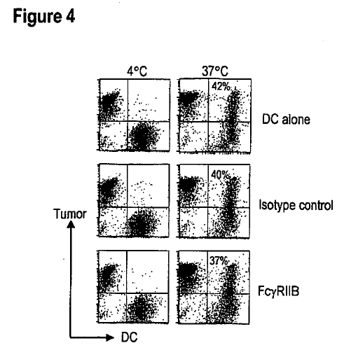

The effect of blockade of FcγRIIB on DC uptake of tumor cells was evaluated. Myeloma tumor cells coated with anti-syndecan-1 antibody were cultured with immature DCs pretreated with either isotype-matched or anti-FcγRIIB antibody. Blockade of FcγRIIB did not alter the uptake of tumor cells by DCs (FIG. 4). In prior studies, phenotypic DC maturation was not observed following uptake of antibody coated myeloma cells by DCs, which may be due to simultaneous engagement of both activating and inhibitory receptors. Indeed, blocking FcγRIIbR with the new antibody (2B6) led to upregulation of DC maturation markers consistent with the data in prior FIG. 2. If the inflammatory cytokine cocktail was added, the DCs could be induced to undergo more complete phenotypic maturation with further upregulation of CD83 (data not shown).

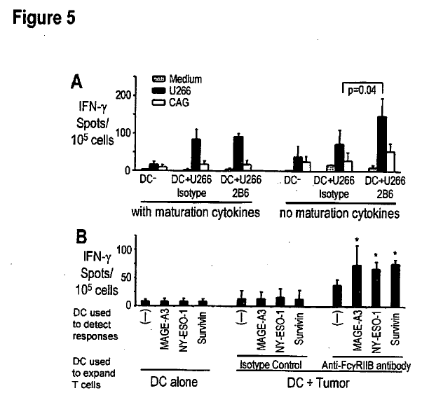

It was also of interest to determine whether blocking FcγRIIB leads to the stimulation of tumor-specific T cells. In prior experiments, generation of anti-tumor immunity by antibody coated tumor loaded DCs required the addition of exogenous maturation stimuli. In contrast, FcγRIIB blockade of DCs leads to enhanced stimulation of the T cells, even in the absence of additional maturation stimuli (FIG. 5A). Indeed, the presentation of tumor antigens by these DCs was comparable to those elicited using DCs that had undergone full maturation using a cytokine cocktail.

To further test if FcγRIIb blockade enhanced the generation of tumor antigen specific T cells, immature DCs from HLA-A2+ individuals were loaded with A2 negative cag myeloma cells, and used to stimulate autologous T cells in the presence or absence of anti-FcγRIIB antibody, as described earlier. Cag cells express high levels of cancer testis antigens MAGE-A3 and NY-ESO-1, and the effects of FcγRIIB blockade on enhancing cross presentation of these antigens by tumor cell loaded DCs could be determined. Stimulation with tumor loaded DCs treated with anti-FcγRIIB antibody (without any additional maturation stimulus) led to enhanced T cell responses to defined A2 restricted epitopes from MAGE-A3, NY-ESO-1, and an overlapping peptide library derived from a shared tumor antigen, survivin (FIG. 5b), in terms of T cell production of interferon-γ.

What is claimed is:

1. A composition for stimulating or enhancing an immune response, the composition comprising
an agent which inhibits signaling via the FcγRIIB receptor; and
an agent which stimulates or enhances signaling via an FcγRI receptor, an FcγRIIa receptor, an FcγRIII receptor, or a combination thereof.

2. The composition of claim 1, wherein said agent, which inhibits signaling via the FcγRIIB receptor is a neutralizing antibody or a fragment thereof.

3. The composition of claim 1, further comprising an adjuvant.

4. A method for stimulating or enhancing an immune response in a subject, comprising the steps of contacting an antigen presenting cell with:
an agent which inhibits signaling via the FcγRIIB receptor; and
an agent which stimulates or enhances signaling via an FcγRI receptor, an FcγRIIa receptor, an FcγRIII receptor, or a combination thereof,
whereby said antigen presenting cell contacts a T lymphocyte and said T lymphocyte stimulates or enhances an immune response in said subject, thereby being a method for stimulating or enhancing an immune response in a subject.

5. The method of claim 4, wherein said antigen presenting cell is a dendritic cell.

6. The method of claim 4, wherein said antigen presenting cells are contacted in vivo with:
said agent which inhibits signaling via the FcγRIIB receptor; or
said agent which stimulates or enhances signaling via an FcγRI receptor, or an FcγRIIa receptor, or an FcγRIII receptor; or
a T lymphocyte: or
a combination thereof.

7. The method of claim 4, wherein said antigen presenting cells are contacted ex vivo with:
said agent which inhibits signaling via the FcγRIIB receptor; or
said agent which stimulates or enhances signaling via an FcγRI receptor, or an FcγRIIa receptor, or an FcγRIIB receptor; or
a T lymphocyte;
or a combination thereof.

8. The method of claim 4, wherein said agent, which inhibits signaling via the FcγRIIB receptor is a neutralizing antibody or a fragment thereof.

9. The method of claim 4, wherein said agent, which stimulates or enhances signaling via an FcγRI receptor, an FcγRIIa receptor, an FcγRIII receptor, or combination thereof, is an immune complex.

10. The method of claim 4, wherein said immune complex comprises a polypeptide or peptide, which is bound to said antibody or antibody fragment.

11. The method of claim 11, wherein said polypeptide or peptide is increasingly or preferentially expressed during disease or infection.

12. The method of claim 4, wherein said antibody or antibody fragment is further bound to a cell.

13. A method for treating, suppressing, or preventing cancer in a subject, the method comprising the steps of contacting an immature dendritic cell with:
an agent which inhibits signaling via the FcγRIIB receptor, and
an agent which stimulates or enhances signaling via an FcγRI receptor, an FcγRIIa receptor, an FcγRIII receptor, or a combination thereof;
whereby said dendritic cell contacts a T lymphocyte and said T lymphocyte stimulates or enhances an immune response against said cancer in said subject, thereby being a method for treating, suppressing, or preventing cancer in a subject.

14. The method of claim 13, wherein said dendritic cells are contacted ex vivo with:
said agent which inhibits signaling via the FcγRIIB receptor; or
said agent which stimulates or enhances signaling via an FcγRI receptor, or an FcγRIIa receptor, or an FcγRIII receptor; or
a T lymphocyte;
or a combination thereof.

15. The method of claim 13, wherein said T lymphocyte stimulates or enhances said immune response via cytolysis.

16. The method of claim 13, wherein said agent, which inhibits signaling via the FcγRIIB receptor is a neutralizing antibody or a fragment thereof.

17. The method of claim 13, wherein said agent, which stimulates or enhances signaling via an FcγRI receptor, an FcγRIIa receptor, an FcγRIII receptor, or combination thereof, is an immune complex.

18. The method of claim 17, wherein said immune complex comprises a polypeptide or peptide, which is bound to said antibody or antibody fragment, and, wherein said polypeptide or peptide is increasingly or preferentially expressed as a function of neoplasia.

19. The method of claim 18, wherein said polypeptide or peptide is increasingly or preferentially expressed prior to the onset of neoplasia.

20. The method of claim 13, wherein said subject has, has had, or is at increased risk for said disease.
